(19) United States
(12) Patent Application Publication
(10) Pub. No.: US 2026/0315635 A1
(43) Pub. Date: Oct. 15, 2026

(54) ANTIMICROBIAL DRYER SHEET

(71) Applicant: The Procter & Gamble Company, Cincinnati, OH (US)

(72) Inventors: Michael Paul FONTAINE, Springboro, OH (US); Kristin Rhedrick WILLIAMS, West Chester, OH (US); Samuel Kimani NJOROGE, Montgomery, OH (US); Jonathan Leslie WEIDENBACHER, Cincinnati, OH (US); Susana E. BORRERO, Mason, OH (US)

(21) Appl. No.: 19/174,227

(22) Filed: Apr. 9, 2025

(51) Int. Cl. A61L 2/20; A61L 2/07; A61L 2/18; A61L 2/26

(52) U.S. Cl. CPC A61L 2/208; A61L 2/07; A61L 2/186; A61L 2/26; A61L 2103/50

(57) ABSTRACT

A method of producing antimicrobial vapor in a laundry dryer is disclosed. Steps include providing a loaded substrate having from about 8 grams to about 10 grams of aqueous hydrogen peroxide thereon; placing a portion of or one or more loaded substrates in the laundry dryer; placing articles of fabric which are desired to be disinfected in the laundry dryer; and operating the laundry dryer in a first cycle, wherein the first cycle has a time period of at least about 15 minutes and an operating temperature of at least about 60 °C.

### FIELD

The present invention relates to methods and systems for providing sanitization or disinfection of fabric articles in an automatic dryer.

### BACKGROUND

Consumers are continually expressing the desire to have soft and fresh smelling clothing that is free of static cling. To obtain such benefits, in laundering it is common for consumers to place dryer sheets along with their clothing in their automatic laundry drying machines. Dryer sheets typically reach most pieces of fabric in the load as they tumble along with the clothing as the automatic laundry drying machine drum rotates.

Generally, conventional dryer sheets are made up of non-woven fibrous substrates which are impregnated or coated with fabric enhancers such as a fabric conditioning active composition, cationic softening agents, antistatic agents, dispersing agents and perfumes. The fabric enhancers are applied to the non-woven fibrous substrate and then dried in an oven or run over chilled rollers so that the dryer sheet is “dry” when ready for use. These dryer sheets may be readily placed in the automatic laundry drying machine with the consumer's clothing.

During the drying process, the fabric enhancers impregnated or coated on the dryer sheet are released and transferred from the dryer sheet to the clothing, either directly from the dryer sheet to the clothing or indirectly by first transferring from the dryer sheet onto to the drum and then from the drum onto the clothing. Examples of dryer sheets may include but are not limited to dryer sheets such as those sold under the tradename BOUNCE DRYER SHEETS manufactured and sold by The Procter & Gamble Company, Cincinnati, Ohio, USA and SNUGGLE DRYER SHEETS manufactured and sold by The Sun Products Corporation, Wilton, Conn., USA.

As a results of the global Covid pandemic, many consumers have been focused on antibacterial products of all sorts. Some manufacturers introduced laundry additives which, in combination with laundry detergent, kill bacteria in the wash. However, there remains no solution for achieving an antibacterial benefit in the dryer.

### SUMMARY

The loaded substrates described herein can provide an antimicrobial benefit for fabric articles in an automatic laundry dryer. A method of producing antimicrobial vapor in a laundry dryer comprises the steps of: providing a loaded substrate comprising from about 8 grams to about 10 grams of aqueous hydrogen peroxide thereon; placing the loaded substrate in the laundry dryer; placing fabric articles which are desired to be disinfected in the laundry dryer; and operating the laundry dryer in a first cycle, wherein the first cycle has a time period of at least about 15 minutes and an operating temperature of at least about 60 °C.

A method for sanitizing fabric articles comprises the steps of: providing a loaded substrate comprising from about 8 grams to about 10 grams of aqueous hydrogen peroxide thereon; applying the loaded substrate to one or more fabric articles; placing the loaded substrate into the laundry dryer; placing the one or more fabric articles which are desired to be disinfected in the laundry dryer; and operating the laundry dryer in a first cycle, wherein the first cycle has a time period of at least about 15 minutes and an operating temperature of at least about 60 °C.

These and other features, aspects, and advantages of various embodiments will become better understood with reference to the following description, figures, and claims.

### BRIEF DESCRIPTION OF THE DRAWINGS

Many aspects of this disclosure can be better understood with reference to the following figures, which illustrate examples according to various embodiments.

It should be understood that the various examples of the of the present disclosure are not limited to that which is illustrated in the figures.

FIG. 1 is a graph depicting dryer cycle temperature versus dryer cycle setting versus time.

FIG. 2 is a graph showing the ppm of hydrogen peroxide vapor versus hydrogen peroxide concentration versus dryer cycle setting versus time.

FIG. 3 is a drawing of an unfolded dryer sheet having fold lines.

FIG. 4 is a perspective view of a folded discrete dryer sheet.

FIG. 5 is a side view of a dryer sheet having a z-fold.

FIG. 6 is a side view of a stack of dryer sheets having z-folds.

FIG. 7 is a perspective view of a plurality of discrete dryer sheets in a stack located within the interior of a carton.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

The term “natural fibers” as used herein, refers to fibers which comprise cellulose-based fibers, bamboo-based fibers, and the like. Natural fibers also refers to nonwoody fibers, such as cotton, abaca, kenaf, sabai grass, flax, esparto grass, straw, jute hemp, bagasse, milkweed floss fibers, and pineapple leaf fibers; and woody, wood, or pulp fibers such as those obtained from deciduous and coniferous trees, including softwood fibers, such as northern and southern softwood kraft fibers; hardwood fibers, such as eucalyptus, maple, birch, and aspen. Pulp fibers can be prepared in high-yield or low-yield forms and can be pulped in any known method, including kraft, sulfite, high-yield pulping methods and other known pulping methods. The natural fibers of the present disclosure may be recycled natural fibers, virgin natural fibers or mixes thereof. Additionally, for good mechanical properties in natural fibers, it can be desirable that the natural fibers be relatively undamaged and largely unrefined or only lightly refined. The fibers can have a Canadian Standard Freeness of at least 200, more specifically at least 300, more specifically still at least 400, and most specifically at least 500.

As used herein “hydrophilic” and “hydrophobic” have meanings as well established in the art with respect to the contact angle of water on the surface of a material. Thus, a material having a water contact angle of greater than about 90 degrees is considered hydrophobic, and a material having a water contact angle of less than about 90 degrees is considered hydrophilic. Compositions which are hydrophobic, will increase the contact angle of water on the surface of a material while compositions which are hydrophilic will decrease the contact angle of water on the surface of a material. Notwithstanding the foregoing, reference to relative hydrophobicity or hydrophilicity between a material and a composition, between two materials, and/or between two compositions, does not imply that the materials or compositions are hydrophobic or hydrophilic. For example, a composition may be more hydrophobic than a material. In such a case neither the composition nor the material may be hydrophobic; however, the contact angle exhibited by the composition is greater than that of the material. As another example, a composition may be more hydrophilic than a material. In such a case, neither the composition nor the material may be hydrophilic; however, the contact angle exhibited by the composition may be less than that exhibited by the material.

Unless otherwise specified, all percentages indicating the amount of a component in a composition represent a percent by weight of the component based on the total weight of the composition. The term “mol percent” or “mole percent” generally refers to the percentage that the moles of a particular component are of the total moles that are in a mixture. The sum of the mole fractions for each component in a solution is equal to 1.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent.

The inventors have found methods/products by which an antimicrobial vapor may be created in the automatic dryer. The antimicrobial vapor can be effective at killing bacteria on articles within the dryer. The antimicrobial effect can lead to more peace of mind for consumers which seek an antimicrobial benefit for their clothing. Additionally, as some microbial entities can contribute to malodor, the methods/products disclosed herein can help reduce and/or eliminate malodor on articles within the dryer.

The creation of antimicrobial vapor within the dryer can be carried out by a variety of methods. For example, in commercial uses, a tank of hydrogen peroxide may be heated and the antimicrobial vapor may be transmitted to the dryer. The amount of antimicrobial vapor may be monitored and carefully metered via control valves and control programming. However, such configurations are not suitable for residential uses.

In residential settings, the vast majority of homeowners don't have space for a hydrogen peroxide tank, heater, and control valves to carefully meter the amount of antimicrobial vapor being provided to their automatic dryer. In such cases, the inventors have found that a nonwoven article, e.g., a dryer sheet, loaded with antimicrobial agent, e.g., hydrogen peroxide, may be utilized. The nonwoven may be loaded with aqueous hydrogen peroxide. In such configurations, the user can simply add the articles to the dryer for which the antimicrobial benefit is sought along with a loaded substrate. It is worth noting the methods/products described herein for use in residential settings may similarly be applied to commercial settings.

There are threshold temperatures and times required for the antimicrobial vapor to be created. For example, a household dryer's air dry or air fluff cycle, where no heat is added, may not generate sufficient antimicrobial vapor to achieve the antimicrobial benefit. Additionally, dryer cycles which run for short periods of time may not generate sufficient antimicrobial vapor to achieve the antimicrobial benefit.

An effective level of antimicrobial vapor within the dryer can be from about 10 ppm to about 40 ppm, preferably greater than about 10 ppm to about 35 ppm, more preferably from about 12 ppm to about 25 ppm, or even more preferably from about 15 ppm to about 20 ppm. In order achieve these levels of antimicrobial vapor, the nonwoven article may be loaded with an aqueous antimicrobial agent, e.g., hydrogen peroxide. There are several factors which can impact the level of antimicrobial vapor generated. Of import, is the time and temperature of the dryer in which the nonwoven article of the present disclosure is place.

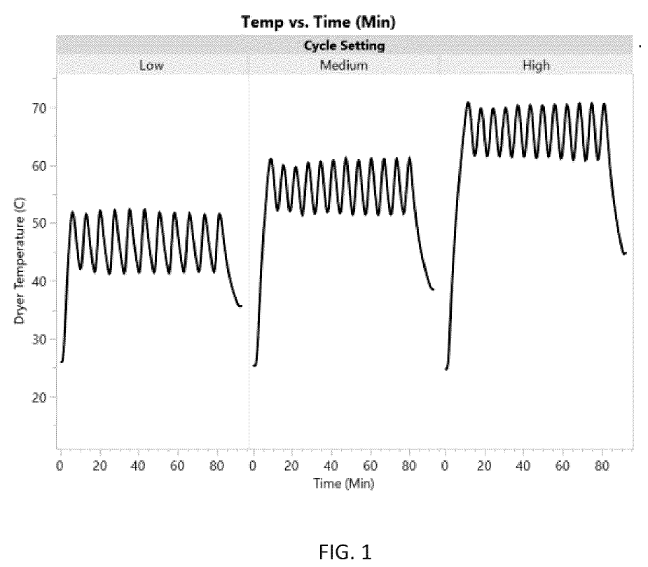

It is believed that the temperature and time of the dryer cycle are inversely proportional. The lower the temperature of the dryer cycle, the more time the articles need to stay in the dryer to achieve the antimicrobial benefit. For example, a loaded substrate when utilized in a dryer cycle of operating at a temperature at least about 60 degrees C, the cycle time may be at least about 15 minutes. However, the same substrate when utilized in a dryer cycle operating at a temperature of less than about 60 degrees C, e.g., about 50 degrees C, the cycle time may be at least about 30 minutes. A graph showing dryer temperature, time and cycle settings is shown in FIG. 1.

The loaded nonwovens of the present disclosure may be utilized in a first cycle of a dryer, wherein the first cycle has an operating time of from between about 15 minutes to about 40 minutes, more preferably from about 15 minutes to about 30 minutes, or even more preferably from about 15 minutes to about 25 minutes. In one specific example, the first cycle time is about 20 minutes.

Additionally, the loaded nonwovens of the present disclosure may be utilized in the first cycle having a first cycle temperature of from between about 50 degrees C to about 80 degrees C, more preferably from about 60 degrees C to about 75 degrees C, or preferably from about 65 degrees C to about 70 degrees C. It is worth noting that the temperature of the dryer should not go above the boiling point of the antimicrobial composition, e.g., hydrogen peroxide.

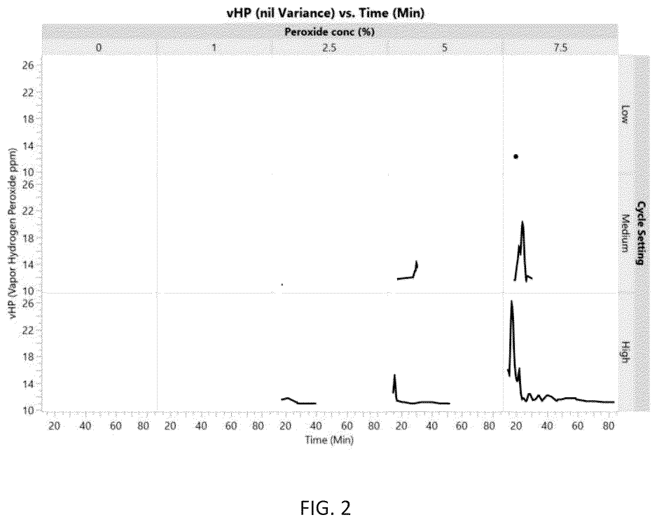

The inventors have surprisingly discovered that while a sufficient amount of antimicrobial vapor can provide the antimicrobial benefit, too much antimicrobial vapor or aqueous peroxide can be irritating to the consumer. For example, too much hydrogen peroxide vapor can cause irritation to the respiratory tract and too high aqueous concentration can cause skin irritation. In order to reduce the likelihood skin irritation, yet provide an effective antimicrobial benefit, aqueous hydrogen peroxide can be formulated at a concentration of from about 2.5 percent to about 7.5 percent, more preferably from about 3 percent to about 6 percent, or even more preferably from about 4 percent to about 5 percent. In order to reduce the risk of generating too much antimicrobial vapor, the nonwoven may be loaded with antimicrobial agent, e.g., aqueous hydrogen peroxide, at a level of up to 6 grams to about 12 grams of aqueous hydrogen peroxide on the nonwoven, more preferably from about 7 grams to about 11 grams, or even more preferably from about 8 grams to about 10 grams. FIG. 2 is a graph showing effective amounts of antimicrobial vapor and its generation as a function of time, dryer cycle, and antimicrobial concentration.

The loaded nonwoven articles of the present disclosure, when utilized in accordance with the times/temperatures/antimicrobial concentrations, described herein provide log reduction of microbial species, leveraging peroxide active materials, of at least 2, more preferably at least 3, even more preferably at least 4, or most preferably at least 5. For example, the loaded nonwoven articles of the present disclosure can provide a log reduction of microbial species, peroxide reactive materials of from between about 5 to about 6. An example of a peroxide reactive material may be Staphylococcus epidermidis, Staphylococcus aureus, salmonella enterica, the influenza virus (H1N1), respiratory syncytial virus, and klebsiella pneumoniae. Additional species are described in U.S. Patent Application Publication No. 2024/0254686. It will be appreciated that antimicrobial efficacy can also be achieved against other target microbes, numerous examples of which will be apparent to those of skill in the art.

As noted, the mechanism for the generation of antimicrobial vapor can be via a loaded nonwoven. Referring to FIG. 1, the nonwoven article 10 may comprise a fibrous material 15. Non-woven materials are defined by ISO standard 9092. The term “non-woven substrate” is used herein in the broadest sense and may mean a sheet of fibers, continuous filaments, or chopped yarns, of any nature or origin, that have been formed into a web by any means, and bonded together by any means, with the exception of weaving or knitting.

When the nonwoven article 10 is placed inside the automatic laundry drying machine along with articles to be dried, and the automatic laundry drying machine is turned on, the heat of the dryer can vaporize the aqueous hydrogen peroxide and create antimicrobial vapor. The antimicrobial vapor, as it is contained within the dryer, is exposed to all the articles within the dryer. This exposure of the articles within the dryer to the antimicrobial vapor is believed to provide an antimicrobial/malodor benefit.

The nonwoven may include but is not limited to those used in dryer sheets such as those sold under the tradename BOUNCE DRYER SHEETS manufactured and sold by The Procter & Gamble Company, Cincinnati, Ohio, USA and SNUGGLE DRYER SHEETS manufactured and sold by The Sun Products Corporation, Wilton, Conn., USA.

The nonwoven may be configured to provide dual benefit to laundered articles. For example, the nonwoven may be designed such that it can release antimicrobial composition, e.g., hydrogen peroxide, as desired, such as when applied directly by a user to an article that will be laundered. In such configurations, the nonwoven may be utilized to provide spot treatment for areas of suspected malodor or areas of microbial growth, e.g., blood stains. In such configurations, the user may spot treat one or more articles as desired and then utilize the nonwoven and/or a new nonwoven in the dryer along with the article or plurality of articles within the dryer to provide antimicrobial vapor to the same in the dryer.

Nonwoven materials of the present disclosure may comprise one or more layers as well as a variety of materials in each of the one or more layers. For example, nonwovens of the present disclosure may be designed to retain aqueous hydrogen peroxide and desorb the same upon application of the substrate to a fabric article. The nonwoven materials of the present disclosure may comprise at least one composition substrate as well as an optional scrubbing substrate. Preferably, the fibers and/or filaments utilized in the nonwoven materials of the present disclosure are oxidation stable.

The composition substrate may comprise nonwoven fibers or paper. Paper substrates are generally the most biodegradable and thus the preferred substrate material for disposability. The composition substrate may comprise any material capable or absorbing and retaining fluid as needed until either pressure is applied, i.e., spot treatment, and/or heat is applied, i.e., via the automatic dryer. The composition substrate may comprise natural and/or synthetic fibres. Fibers useful in the composition layer include those that are naturally occurring (modified or unmodified), as well as synthetically made fibers.

As noted, ensuring that the fibers do not react with the hydrogen peroxide is preferable as more reactive fibers can break down the peroxide into water thereby reducing the antimicrobial efficacy of the nonwoven article. For example, when choosing fibers for the nonwoven article, selection is preferably done with consideration to the life cycle of a consumer product, e.g., from manufacture, to shipping, to stocking on store shelves, and also time on shelf before purchase. Some suitable examples of low reactive or non-reactive fibers include pulp fibers, Northern Softwood Kraft fibers, Eucalyptus fibers, Sothern Softwood Kraft fibers, high density polyethylene, low density polyethylene, polypropylene, polyethylene terephthalate or combinations thereof. Preferably the fibers and/or filaments of the nonwoven article are selected such that minimal or no reaction with the aqueous hydrogen peroxide occurs. For example, the fibers and/or filaments may be selected such that the hydrogen peroxide loading and concentrations provided herein are maintained for at least 3 months, more preferably 6 months, or most preferably for over 1 year.

Where synthetic constituent materials are utilized, thermoplastic fibers can be made from a single polymer (monocomponent fibers) or can be made from more than one polymer (e.g., bicomponent fibers). As used herein, “bicomponent fibers” refers to thermoplastic fibers that comprise a core fiber made from one polymer that is encased within a thermoplastic sheath made from a different polymer. The polymer comprising the sheath often melts at a different, typically lower, temperature than the polymer comprising the core. As a result, these bicomponent fibers can provide thermal bonding due to melting of the sheath polymer, while retaining the desirable strength characteristics of the core polymer.

Suitable bicomponent fibers for use in the present invention can include sheath/core fibers having polyethylene/polypropylene, polyethylene/polyethylene terephthalate or any combination thereof. These bicomponent fibers can be concentric or eccentric. As used herein, the terms “concentric” and “eccentric” refer to whether the sheath has a thickness that is even, or uneven, through the cross-sectional area of the bicomponent fiber. Eccentric bicomponent fibers can be desirable in providing more compressive strength at lower fiber thicknesses. Preferred bicomponent fibers comprise a copolyolefin bicomponent fiber comprising less than about 81% polyethylene terephthalate core and a less than about 51% copolyolefin sheath. Such a preferred bicomponent fiber is commercially available from the Hoechst Celanese Corporation, in New Jersey, under the trade name CELBOND® T-255. The amount of bicomponent fibers will preferably vary according to the density of the material in which it is used.

Any suitable method for making the nonwoven articles of the present disclosure may be utilized. For example, the nonwoven article may be wetlaid as is known in the art. As another example, the nonwoven article of the present disclosure may be carded and bonded as is known in the art. In yet another example, the nonwoven article of the present disclosure may comprise multiple layers which utilize different processes for each of the layers. Where the nonwoven articles comprise more than one layer, any suitable method may be utilized to join the multiple layers together. Methods for preparing thermally bonded fibrous materials are described in U.S. Pat. No. 5,607,414 (Richards et al), issued Mar. 4, 1997; and U.S. Pat. No. 5,549,589 (Homey et al) issued Aug. 27, 1996 (see especially columns 9 to 10). Such foams and methods for their preparation are described in U.S. Pat. No. 5,550,167 (DesMarais), issued Aug. 27, 1996; and U.S. Pat. No. 5,563,179 (Desmarais et al.), issued Oct. 8, 1996.

Additional methods of making nonwovens are well known in the art. Generally, these nonwovens can be made by air-laying, water-laying, meltblowing, coforming, spunbonding, or carding processes in which the fibers or filaments are first cut to desired lengths from long strands, passed into a water or air stream, and then deposited onto a screen through which the fiber-laden air or water is passed. In one example, the nonwoven article may comprise a carded, spunlaced partially hydrophobic substrate.

In addition to the fibers used to make the nonwoven material, the nonwoven material can comprise other components or materials added thereto as known in the art, including binders, dry strength and lint control additives.

The composition substrate may comprise a lofty substrate, more preferably a batting substrate. Batting is defined according to the TAPPI Association of the Nonwoven Fabrics Industry as a soft bulky assembly of fibres. The batting may comprise either a combination of fibers comprising the above-listed materials or fibers which themselves comprise a combination of the above-listed materials. In any instance, side-by side configuration, core-sheath configuration, or solid single component configuration, the fibers of the batting may exhibit a helical or spiral or crimped configuration, particularly the bicomponent type fibers. Furthermore, the fibers of the batting may be of varying sizes, i.e., the fibers of the batting may comprise fibers having different average thicknesses. Also, the cross section of the fibers can be round, flat, oval, elliptical or otherwise shaped.

Preferred fibers of the composition layer are those susceptible of heat sealing. The composition substrate may comprise a combination of single component and bicomponent fibers. More specifically it is preferred that the composition substrate comprises polyester single component fibers and polyester core, polyethylene sheath bicomponent fibers.

In addition to reduced, and preferably no reaction with the antimicrobial composition, the fibers for the nonwoven article may also be selected such that they have the right level of hydrophilicity. For example, if hydrophobic fibers are selected, the antimicrobial composition will likely run off the nonwoven article. This can cause a negative consumer perception as every use of the nonwoven article would mean antimicrobial composition being spilled once the nonwoven article is removed from its package.

In contrast, if too hydrophilic, then the antimicrobial composition will be more difficult to extract from the substrate. It is worth noting that fibers may be treated with hydrophilic compositions and/or hydrophobic compositions to achieve the desired characteristics of the nonwoven article.

Some suitable examples of hydrophilic compositions include non-ionic surfactants including esters, amides, carboxylic acids, alcohols, ethers—polyoxyethylene, polyoxypropylene, sorbitan, ethoxylated fatty alcohols, alyl phenol polyethoxylates, lecithin, glycerol esters and their ethoxylates, and sugar-based surfactants (polysorbates, polyglycosides). Other suitable examples include anionic surfactants including sulfonates, sulfates, phosphates, alkali metal salts of fatty acids, fatty alcohol monoesters of sulfuric acid, linear alkyl benzene sulfonates, alkyl diphenyloxide sulfonates, lignin sulfonates, olefin sulfonates, sulfosuccinates, and sulfated ethoxylates of fatty alcohols. Other suitable examples include cationic surfactants including amines (primary, secondary, tertiary), quaternary ammoniums, pyridinium, quaternary ammonium salts-QUATS, alkylated pyridinium salts, alkyl primary, secondary, tertiary amines, and alkanolamides. Other suitable examples include zwiterionic surfactants including amino acids and derivatives, amine oxide, betaines, and alkyl amine oxides. Other suitable examples include polymeric surfactants including polyamines, carboxylic acid polymers and copolymers, EO/PO block copolymers, ethylene oxide polymers and copolymers, and polyvinylpyrrolidone. Other suitable examples include silicone surfactants including dimethyl siloxane polymers with hydrophile. And other suitable examples include perfluorocarboxylic acid salts and fluorosurfactants.

Some suitable examples of hydrophobic compositions include fluorinated or perfluorinated polymers; silicones; fluorochemicals; zirconium compounds; oils; latexes; waxes; crosslinking resins; and blends thereof; fluorochemical urethanes, ureas, esters, ethers, alcohols, epoxides, allophanates, amides, amines (and salts thereof), acids (and salts thereof), carbodiimides, guanidines, oxazolidinones, isocyanurates, and biurets; nanostructured particles selected from fumed silica, hydrophobic titania, zinc oxide, nanoclay, and mixtures thereof; fats and oils, glycerol derivatives; hydrophobic silicones or suitable combinations thereof.

The nonwoven article preferably has a basis weight of from about 20 grams per square meter (“gsm”) to about 200 gsm, more preferably, at least about 20 gsm and more preferably less than about 150 gsm, more preferably the base weight is in the range of about 20 gsm to about 120 gsm, and most preferably from about 30 gsm to about 110 gsm. The nonwoven article may have any caliper. Typically, when the nonwoven is made by hydroentangling, the average substrate caliper is less than about 1.2 mm at a pressure of about 0.1 pounds per square inch. More preferably the average caliper of the substrate is from about 0.1 mm to about 1.0 mm at a pressure of about 0.1 pounds per square inch (about 0.007 kilograms per square meter). The substrate caliper is measured according to standard EDANA nonwoven industry methodology, reference method #30.4-89.

In addition to the composition substrate, the nonwoven articles of the present disclosure may further comprise a scrubbing substrate. The scrubbing substrate can provide a comparatively more abrasive surface than the composition substrate and as such is useful in scrubbing dirt, debris, and/or residue/soil, especially tough to remove residue/soil, from fabrics.

The scrubbing substrate may comprise an abrasive net of fibres, otherwise known as a scrim. By the term ‘net’ it is meant a structure made directly from melts or fibres which are at least 0.2 mm long and are held together by systems other than hydrogen bonding. The fibres may be selected from natural and/or synthetic, filaments or stands or mixtures thereof as long as the resulting web provides a surface which is more abrasive than the composition substrate. Preferred fibres are selected from those of synthetic organic origin listed herein, more preferably from polymeric synthetic organic origin and thermoplastic polymers. The fibres are preferably selected from polyamide, polyethylene, polypropylene fibres and mixtures thereof.

The scrim is typically wrapped around a secondary structure to provide some bulk. The shape of the holes in the scrim can include, but is not limited to, a variety of shapes such as squares, rectangles, diamonds, hexagons or mixtures thereof. Typically, the smaller the area composed by the holes in the scrim the greater the scrubbing ability. This is primarily due to the fact that there are more points where the scrim material intersects, as it is these intersection points that will contact the fabric. An alternative to wrapping netting or scrim is to apply molten extruded polymers directly onto a secondary structure such as a non-woven. Upon solidifying the polymer would create high point stiffer material as compared to the secondary non-woven, and thereby provides scrubbing ability.

The scrubbing substrate may be a low density, e.g., about 0.00005 g/cm³ to about 0.1 g/cm³, preferably from about 0.001 g/cm³ to about 0.09 g/cm³ and a thickness of from about 0.04 inches to about 2 inches at 5 gms/in², The scrubbing substrate may comprise fibers of polyethylene terephthalate (PET), and bicomponent sheath core fibers made from polyethylene (PE) and polyethylene terephthalate (PET).

The fibers may be randomly arranged but are preferably ordered. The net may be made using any known process, including those described above for preparing nonwoven substrates. For example, the fibers may be arranged in an open lattice wherein the fibers are, for example, knitted or extruded together to form the net.

The scrubbing substrate may be embossed, such that the scrubbing substrate exhibits a pattern comprised primarily of male projections. On the other hand, the scrubbing substrate may be debossed, meaning that the web exhibits a pattern comprised primarily of female capillary networks. As with the composition substrate it is highly preferred that the scrubbing substrate is flexible and even more preferably the substrate is also resilient meaning that once applied pressure has been removed the substrate regains it's original shape. Similarly, the scrubbing substrate may comprise one or more layers. The typical basis weight for flexural stiff materials suitable for use as the scrubbing substrate range from 20 to 150 gsm, for instance 30 to 125 gsm.

For example, the scrubbing substrates of the present Preferred film materials are pervious to liquids, and in particular liquids containing soils, and yet are non-absorbent and have a reduced tendency to allow soiled liquids to pass back through their structure and rewet the fabrics being cleaned. Preferably the film material comprises a plurality of protrusions extending outwardly from the film surface and away from the body of the cleaning pad. Alternatively, or additionally, the film may comprise a plurality of apertures.

The composition and scrubbing substrates are preferably attached, potentially reversibly attached, to one another. The point of attachment can be at any point over the surface of the nonwoven material, as long as the scrubbing substrate and composition substrate are attached to one another. Even more preferably the cleaning and scrubbing substrates are attached to one another around the perimeter of the scrubbing and/or cleaning substrates. The substrates may be attached to one another using any commonly known method, for example using heat sealing, adhesive, ultrasonic sealing, stitching and combinations thereof. Preferably the substrates are attached to one another using heat sealing. Even more preferably the substrates are attached to one another, by a combination of heat sealing around the perimeter of the substrate and dot heat sealing, preferably in a pattern, across the surface area of the wipe. This latter method of heat sealing is described in more detail in the embodiment comprising a partially or fully water impermeable membrane. Where heat sealing is used, it is necessary that the composition and/or scrubbing substrate comprise thermoplastic polymers. The greater the content of thermoplastic polymers the stronger is the sealing.

It is believed that the nonwoven article delivers the antimicrobial benefit within the dryer via diffusion. As noted herein, there is a relatively high concentration of antimicrobial composition on the nonwoven article and no antimicrobial composition vapor in the headspace of the dryer, at time equal to zero of the dryer cycle. As the dryer increases the internal temperature of the drum, antimicrobial vapor is created which provides some of the antimicrobial benefit. However, it is further believed that some of the antimicrobial benefit is provided via contact with the nonwoven article. In order to ensure adequate contact with the laundry articles within the dryer, the nonwoven article should have a sufficient surface area.

Suitable surface area for the nonwoven articles is from about 25 square cm to about 400 square inches, more preferably 50-200 sq inches, and most preferably 100 sq inches. For those configurations where the nonwoven article comprises 10g aqueous peroxide preferred execution, that puts the fluid gsm at around 0.1 gsm or a range of 0.05 gsm-0.2 gsm.

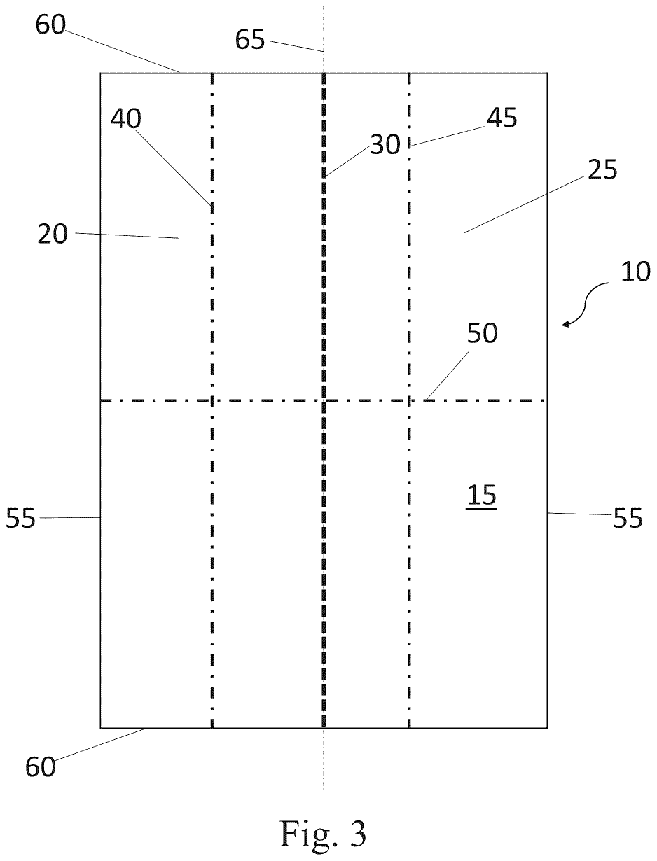

FIG. 3 is a drawing of an unfolded nonwoven article 10 having fold lines. The loaded substrate 10 may have at least a first portion 20, a second portion 25, and a frangible boundary 30 between the first portion 20 and the second portion 25. The frangible boundary 30 is indicated in FIG. 3 by a heavy dashed line. The frangible boundary 30 may comprise a plurality of perforations. The loaded substrate 10 may be substantially rectangularly shaped. The l nonwoven article 10 may have an axis 65. The nonwoven article 10 may have two opposing long edges 55 substantially parallel to and on opposing sides of said axis 65 defining a width there between and two opposing short edges 60 substantially orthogonal to said axis 65 defining a length there between. The frangible boundary 30 may be along or coincident with an axis 65 substantially parallel to the long edges 55, as indicated in FIG. 3 by a thin dot dash line. The nonwoven article 10 may be folded along a first fold line 40 and a second fold line 45 substantially parallel to the first fold line 40 to have a z-fold. The nonwoven article 10 may be folded along a third fold line 50 substantially perpendicular to the first fold line 40 to have a c-fold. The first fold line 40, second fold line 45, and third-fold line 50 are indicated in FIG. 3 by heavy dash-dot lines.

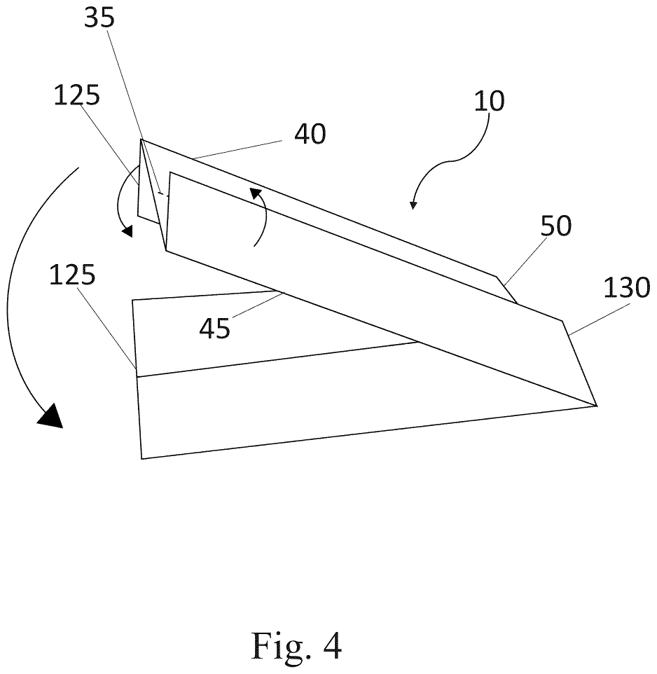

FIG. 4 is an example of a folded discrete nonwoven article 10. The nonwoven article 10 may be folded as indicated by the direction of the arrow along a first fold line 40 and further folded as indicated by the direction of the arrow along a second fold line 45 substantially parallel to the first fold line 40 to have a z-fold. The nonwoven article 10 may be further folded as indicated by the direction of the arrow along a third fold line 50 substantially perpendicular to the first fold line 40 to have a c-fold. The nonwoven article 10 may be folded along a third fold line 50 substantially perpendicular to the first fold line 40 to have a c-fold, thereby creating a dryer sheet driver end 130 at the third fold 50 and a dryer sheet operator end 125. The frangible boundary 30 may comprise a plurality of perforations 35, as indicated in FIG. 4 by a dashed line.

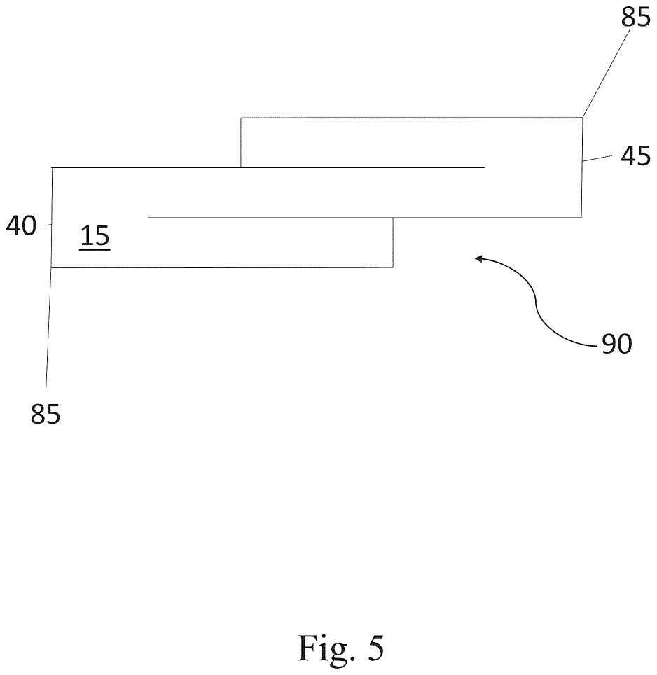

FIG. 5 is a drawing of nonwoven article 10 having a z-fold 90. As shown, the nonwoven article 10 may be folded along a first fold line 40 to be have a c-fold 85 along one portion of the nonwoven article 10. The nonwoven article 10 may further be folded along a second fold line 45 substantially parallel to the first fold line 40 to have a z-fold 90. In operation, a z-fold 90 is comprised of two c-folds 85.

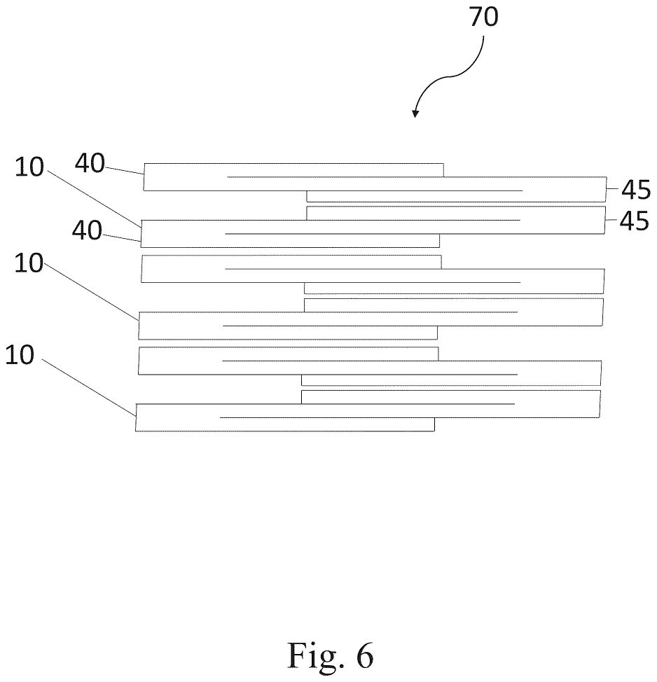

FIG. 6 shows a stack 70 of loaded substrates 10, wherein each nonwoven article 10 has a z-fold 90. Each loaded substrate 10 may be placed on top of, but is not interleaved, with the nonwoven article 10 immediately above and/or below. Alternatively, the plurality of loaded substrates 10 may be in a stack 70 wherein each nonwoven article 10 is interleaved with the nonwoven article 10 immediately above and/or below. The stack 70 of folded loaded substrates 10 may be vertically stacked, such that each nonwoven article 10 may lie on top of the nonwoven article 10 immediately below, as shown in FIG. 6. Alternatively, the stack 70 of folded nonwoven articles 10 may be horizontally stacked, such that each nonwoven article 10 may lie adjacent with the nonwoven article 10 immediately to the left and/or to the right.

### Perfume

The nonwoven article 10 may comprise perfume. For example, each of the first portion 20 and the second portion 25 may comprise at least about 0.013 grams of a perfume, at least about 0.026 grams of perfume or even more preferably at least about 0.112 grams of a perfume. As another example, each of the first portion 20 and the second portion 25 may comprise at least about 0.003 grams of an encapsulated perfume, more preferably at least about 0.006 grams of an encapsulated perfume, even more preferably at least about 0.01 grams of an unencapsulated perfume, even more preferably at least about 0.02 grams of unencapsulated perfume, even more preferably at least about 0.022 grams of unencapsulated perfume, even more preferably at least about 0.09 grams of an unencapsulated perfume. The nonwoven article 10 may comprise perfume to provide an olfactory aesthetic benefit and/or to serve as a signal that the nonwoven article 10 is effective. Providing a greater quantity of perfume may provide the benefit of additional fresh smell.

The nonwoven article 10 may have at least a first portion 20 and a second portion 25 wherein each of the first portion 20 and the second portion 25 is free or substantially free of a perfume.

The perfume ingredients of the present disclosure can be the conventional ingredients known to one skilled in the art. The perfume ingredients of the present disclosure may be incorporated into the dryer sheet 10 as an unencapsulated free perfume oil, or neat perfume. The perfume ingredients of the present disclosure may be incorporated into the nonwoven article 10 as a perfume encapsulated into a microcapsule to form a perfume microcapsule, or encapsulated perfume. The perfume ingredients of the present disclosure may be provided as a combination of neat and encapsulated perfumes.

The term “unencapsulated perfume” is used herein in the broadest sense and may mean a composition comprising free perfume ingredients wherein the free perfume ingredients are neither absorbed onto or into a perfume carrier (e.g., absorbed on to zeolites or clays or cyclodextrin) nor encapsulated (e.g., in a perfume microcapsule, or encapsulated). An unencapsulated perfume ingredient may also comprise a pro-perfume, provided that the pro-perfume is neither absorbed nor encapsulated. Non-limiting examples of suitable perfumes ingredients include blooming perfumes, perfume oils, and perfume raw materials comprising alcohols, ketones, aldehydes, esters, ethers, nitriles alkenes, and mixtures thereof. Non-limiting examples of blooming perfume ingredients that may be useful in the products of the present invention are given in U.S. Patent Publication 2005/0192207A1 . The loaded substrate 10 may have at least a first portion 20 and a second portion 25 wherein each of the first portion 20 and the second portion 25 is free or substantially free of an unencapsulated perfume.

The term “encapsulated perfume” is used herein in the broadest sense and may include the encapsulation of perfume or other materials or actives in small capsules (i.e., microcapsules), typically having a diameter less than about 300 microns. These microcapsules may comprise a spherical hollow shell of water insoluble or at least partially water insoluble material, typically polymer material, within which the active material, such as perfume, is contained. The shell of the microcapsule may comprise an aminoplast resin. Aminoplast resins are the reaction products of one or more amines with one or more aldehydes, typically formaldehyde. Non-limiting examples of suitable amines include urea, thiourea, melamine and its derivatives, benzoguanamine and acetoguanamine and combinations of amines. Suitable cross-linking agents (e.g., toluene diisocyanate, divinyl benzene, butane diol diacrylate etc.) may also be used and secondary wall polymers may also be used as appropriate, e.g., anhydrides and their derivatives, particularly polymers and co-polymers of maleic anhydride as disclosed in U.S. Patent Publication 2004/0087477A1. The shell of the microcapsule may comprise urea-formaldehyde, melamine-formaldehyde, or combinations thereof.

The encapsulated perfume may comprise a friable perfume microcapsule. Friability refers to the propensity of the microcapsule to rupture or break open when subjected to direct external pressures or shear forces. As disclosed herein, a microcapsule is “friable” if, while attached to fabrics treated therewith, the microcapsule can be ruptured by the forces encountered when the capsule-containing fabrics are manipulated by being worn or handled (thereby releasing the contents of the capsule). Friable perfume microcapsules can be attractive for use in dryer-activated products because not only do the friable perfume microcapsules enable top-note scent characters to deposit easily onto fabrics after the drying process, but they also allow the consumer to experience these scent types throughout the day while wearing their article of clothing. Friable perfume microcapsules rupture and release perfume by a mechanical means (e.g., friction), not a chemical means (e.g., water hydrolysis). Minimal fracture pressure is typically needed to break the structure such as normal everyday physical movements such as taking off a jacket; pulling a shirt off; or taking off/putting on socks. Non-limiting examples of perfume microcapsules suitable as an encapsulated perfume are available in the following references: U.S. Pat. Nos. 6,645,479; 6,200,949; 4,882,220; 4,917,920; 4,514,461; 4,234,627; 2003/215417 A1; 2003/216488 A1; 2003/158344 A1; 2003/165692 A1; 2004/071742 A1; 2004/071746 A1; 2004/072719 A1; 2004/072720 A1; 2003/203829 A1; 2003/195133 A1; 2004/087477 A1; 2004/0106536 A1 and EP Patent Publication 1393706 A1 . The perfume microcapsule may encapsulate a blooming perfume composition, wherein the blooming perfume composition comprises blooming perfume ingredients. A non-limiting example of suitable dryer sheets comprising a perfume microcapsule is disclosed in U.S. Pat. No. 5,425,887.

The first portion 20 and/or second portion 25 can be free or substantially free of an encapsulated perfume. Alternatively, the first portion 20 and second portion 25 may comprise at least about 0.022 grams of an encapsulated perfume.

The encapsulated perfume may comprise a moisture-activated microcapsule, such as those microcapsules wherein the shell of the microcapsule comprises cyclodextrin, e.g., beta-cyclodextrin. The encapsulated perfume may comprise one or more free perfume ingredients either absorbed onto or into a perfume carrier (e.g., zeolites, clays, cyclodextrin, and combinations thereof). The first portion 20 and second portion 25 may comprise at least about 0.022 grams of an encapsulated perfume, wherein the encapsulated perfume comprises a moisture-activated microcapsule and/or a friable microcapsule. The first portion 20 and/or the second portion 25 can be free of a moisture-activated microcapsule.

The encapsulated perfume may comprise a heat-activated microcapsule. As defined herein, a heat-activated microcapsule is one that ruptures or otherwise releases the encapsulated composition by body heat and/or by the heat in a machine dryer. It is worth noting that the loaded substrates of the present disclosure may comprise a combination of microcapsules selected from friable perfume microcapsule, a moisture-activated microcapsule, a heat-activated microcapsule, and combinations thereof. Non-limiting examples of additional microcapsules include wax microcapsule such as those described in U.S. Pat. No. 5,246,603 and starch-based microcapsule also described in U.S. Pat. No. 5,246,603.

The unencapsulated perfume, encapsulated perfume, or combination thereof, may be incorporated into the nonwoven article 10 by adding it to the aqueous antimicrobial composition before the aqueous antimicrobial composition is added to the loaded substrate 10. The unencapsulated perfume, encapsulated perfume, or combinations thereof, may be coated on the loaded substrate 10 by spraying means.

### Optional Ingredients

The loaded substrate 10 may comprise additional optional ingredients. As used herein, an optional ingredient means any material that performs a function or delivers a benefit, such as modifying the physical or chemical properties of the treated material (e.g., fabric).

Suitable optional ingredients may include, but are not limited to: perfumes, softening agents, soil release agents, anti-static agents, crisping agents, water/stain repellents, stain release agents, refreshing agents, disinfecting agents, wrinkle resistant agents, wrinkle release agents, odor resistance agents, malodor control agents, abrasion resistance and protection agents, solvents, insect/pet repellents, wetting agents, chlorine scavenging agents, optical brighteners, UV protection agents, skin/fabric conditioning agents, skin/fabric nurturing agents, skin/fabric hydrating agents, color protection agents, dye fixatives, dye transfer inhibiting agents, silicones, preservatives and anti-microbials, fungicides, fabric shrinkage-reducing agents, brighteners, hueing dyes, bleaches, chelants, antifoams, anti-scum agents, whitening agents, catalysts, cyclodextrin, zeolite, petrolatum, glycerin, triglycerides, vitamins, other skin care actives such as aloe vera, chamomile, shea butter and the like, mineral oils, and combinations thereof. In an embodiment, the loaded substrate 10 may comprise one or more optional ingredients as unencapsulated ingredients. In an embodiment, the loaded substrate 10 may comprise one or more optional ingredients wherein the optional ingredients may be encapsulated in microcapsules. In an embodiment, the loaded substrate 10 may comprise odor control agents (such as cyclodextrin, metal salts, and zeolites), wrinkle control agents, antimicrobial agents, fungicides, preservatives, insect repellents, or combinations thereof. In an embodiment, the loaded substrate 10 may be free or substantially free of one more of the above identified optional ingredients.

### Configuration of Dryer Sheet

### Frangible Boundary

Each nonwoven article 10 may have a frangible boundary 30 between the first portion 20 and the second portion 25. As used herein, the frangible boundary 30 is a weakened boundary where consumers may separate the nonwoven article 10 into the first portion 20 and the second portion 25 by placing tension on the frangible boundary 30 such that the first portion 20 becomes detached from the second portion 25 along the frangible boundary 30. The frangible boundary 30 may comprise a plurality of perforations 35. The frangible boundary 30 may comprise a row of perforations 35 a plurality of cuts, a plurality of apertures or punch out perforations 35 or combinations thereof. Having the first portion 20 and the second portion 25 able to be detached from one another by a frangible boundary 30 may allow for consumers to choose between using an entire nonwoven article 10 or using one portion of a nonwoven article 10, depending on the size of the load in the automatic laundry drying machine. Additionally, the frangible boundary 30 allows for users to utilize a portion of the loaded substrate for spot treatment while reserving the remainder for use in an automatic dryer.

The frangible boundary 30 may be located centrally on the nonwoven article 10. The nonwoven article 10 may be substantially rectangularly shaped. The nonwoven article 10 may have two opposing long edges 55 substantially parallel to and on opposing sides of said axis 65 defining a width there between and two opposing short edges 60 substantially orthogonal to said axis 65 defining a length there between. A rectangularly shaped nonwoven article 10 is familiar to consumers and is convenient to organize when storing a plurality of nonwoven article 10. The frangible boundary 30 may be along or coincident with an axis 65 substantially parallel to the long edges 55. As a general habit of holding substantially rectangularly shaped books and sheets by the two long edges 55 so that the length is vertical to the consumer, having the frangible boundary 30 along or coincident with an axis 65 substantially parallel to the long edges 55 can provide the benefit that consumers will not have to change this habit to separate the first portion 20 from the second portion 25 of the nonwoven article 10 because the nonwoven article 10 will unfold so that the long edges 55 are vertical to the consumer. Alternatively, the frangible boundary 30 may be substantially parallel to the short edges 60. Having the frangible boundary 30 substantially parallel to the short edges 60 may provide the benefit of a shorter length to apply tension to when detaching the first portion 20 from the second portion 25. The nonwoven article 10 may have more than one frangible boundary 30. The nonwoven article 10 may be substantially rectangularly shaped as shown in FIG. 3, however, one of skill in the art will recognize that the shape of the nonwoven article 10 is not so limited. Other suitable nonwoven article 10 shapes may include but are not limited to substantially squared, substantially circular, and substantially triangular.

### Folding

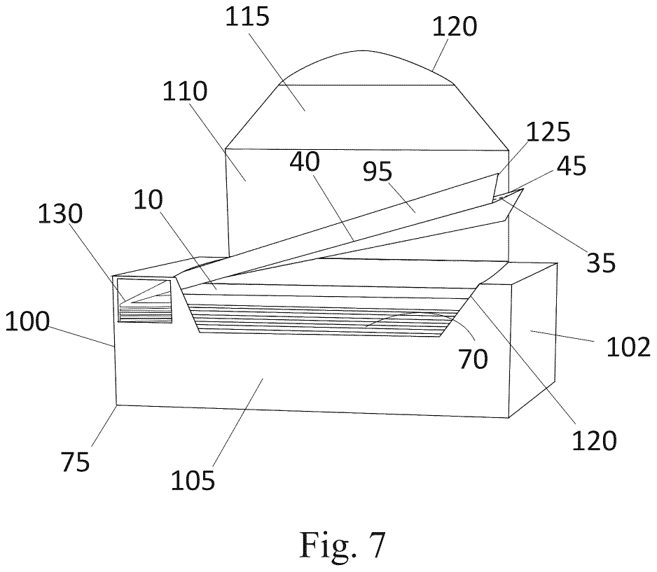

The nonwoven article 10 may be folded. Folding enables the nonwoven article 10 to take up less planar space in a consumer's storage area. The nonwoven article 10 may be folded along a first fold line 40 to have a c-fold 85. The nonwoven article 10 may be folded along a second fold line 45 substantially parallel to the first fold line 40 to have a z-fold 90. The nonwoven article 10 may be folded along a second fold line 45 not substantially parallel to the first fold line 40. In operation, a z-fold 90 is comprised of two c-folds 85, as shown in FIG. 5. Each c-fold 85 may be along a portion of the nonwoven article 10. The nonwoven article 10 may be folded along a third fold line 50 substantially perpendicular to the first fold line 40 to have a c-fold 85. The nonwoven article 10 may be folded along a third fold line 50 substantially perpendicular to the second fold line 45. The nonwoven article 10 may be folded in half along a third fold line 50 substantially perpendicular to the first fold line 40 to have a c-fold 85. The nonwoven article 10 may first be folded as indicated by the direction of the arrow in FIG. 2 along the first fold line 40 and further folded as indicated by the direction of the arrow in FIG. 2 along the second fold line 45 to have a z-fold 90 as shown in FIGS. 2 and 3. The nonwoven article 10 having a z-fold 90 may then be folded as indicated by the direction of the arrow in FIG. 2. along a third fold line 50 to have a c-fold 85. Alternatively, the nonwoven article 10 may first be folded along the third fold line 50 to have a c-fold 85. The nonwoven article 10 having a c-fold 85 may then be folded along the first fold line 40 and further folded along the second fold line 45 to have a z-fold 90. This configuration provides the benefit of the consumer being able to see the frangible boundary 30 when the nonwoven article 10 is folded. The nonwoven article 10 may be folded along a third fold line 50 substantially perpendicular to the first fold line 40 to have a c-fold 85, thereby creating a nonwoven article 10 driver end 130 at the third fold 50 and a nonwoven article 10 operator end 125, as shown in FIGS. 4 and 7.

However, one of skill in the art will recognize that the folding of the loaded substrate 10 is not so limited. The loaded substrate 10 may be folded along any one of the first fold line 40, the second fold line 45, the third fold line 50, and combinations thereof, to have a c-fold, a tri-fold, a z-fold, a double parallel fold, a roll fold, a double gate fold, a 3-panel gate fold, and any combinations thereof.

The loaded substrate 10 may first be folded along a first fold line 40 and along a second fold line 45 substantially parallel to the first fold line 40 to have a z-fold 90. The first fold line 40 and second fold line 45 may be substantially parallel to the frangible boundary 30. The first fold line 40 and the second fold line 45 may be along or coincident with an axis 65 substantially parallel to the long edges 55 of the loaded substrate 10. The loaded substrate 10 may then be folded along a third fold line 50 substantially perpendicular to the first fold line 40 to have a c-fold 85. In such configuration, the loaded substrate 10 can use less planar space in a consumer's storage area and can provide the benefit of a consumer easily picking up the loaded substrate 10 by one of the long edges 55. The loaded substrate 10 may then easily be retrieved from a package 75 and unfolded by first unfolding the loaded substrate 10 along the third fold line 50 to remove the c-fold 85. The loaded substrate 10 may then be further unfolded by unfolding the loaded substrate 10 along the second fold line 45 and along the first fold line 40 to remove the z-fold 90 to have an unfolded dryer sheet 10 as shown in FIG. 1. The loaded substrate 10 may then be separated by placing tension on the frangible boundary 30 such that the first portion 20 becomes detached from the second portion 25 along the frangible boundary 30. Alternatively, the d loaded substrate 10 may be left with the first portion 20 and the second portion 25 attached to one another. By folding the loaded substrate 10 first to have a z-fold 90 and then folding the folded loaded substrate 10 to have a c-fold 85, as shown in FIG. 2, the frangible boundary 30 can be protected from separating prematurely in handling.

### Plurality of Dryer Sheets

The plurality of discrete loaded substrates 10 may be in a stack 70. The plurality of loaded substrates 10 may be placed on top of each other but may not be interleaved, with the loaded substrates 10 immediately above and/or below. Conversely, the plurality of loaded substrates 10 may interleaved with the loaded substrates 10 immediately above and/or below. The stack 70 of folded loaded substrates 10 may be vertically stacked, such that each loaded substrate 10 may lie on top of the loaded substrates 10 immediately below.

Where the plurality of discrete nonwoven articles 10 are first folded to have a z-fold 90 and then folded to have a c-fold 85 such as that shown in FIG. 2, the stack 70 of folded nonwoven articles 10 may be vertically stacked, as shown in FIGS. 6 and 7, such that each nonwoven article 10 may lie on top of the nonwoven article 10 immediately below. As shown in FIG. 7, the nonwoven articles 10 may be in an orientation within the package 75 in which a consumer may curve her index finger under the top flap 95 and her thumb over the top flap 95 to pinch the top flap 95 of the nonwoven article 10 to easily pull the nonwoven article 10 out of the package 75.

The package 75 may be in the shape of a rectangular or square box. The package 75 may have sharp angular edges or smoothed edges. Suitable carton materials may include cardboard and or paper materials and are commonly known and used for storing nonwoven articles 10. Depending upon the number of nonwoven articles 10 to be stored within the package 75 and the nonwoven article 10 dimensions, the package 75 can have varying dimensions. Non-limiting examples of suitable package dimensions are described in U.S. Pat. No. 5,310,057.

The package 75 may have a front panel 105 and an opposing back panel, a left side panel 100 and an opposing right side panel 102, a top panel 110 and an opposing bottom panel. The package 75 may comprise a line of weakness 120 which allows for easy opening of the package 75 to access the interior contents. The line of weakness 120 may comprise a plurality of slits or cuts into the package 75. The line of weakness 120 may be formed from the top panel 110 and optionally one or more side panels 100, 102 of the package 75. The line of weakness 120 may be formed only of one or more of the side panels 100, 102. It should be understood that the line of weakness 120 can be formed in any one or more adjacent panels such that a recloseable access flap 115 is formed from the portion of the panel(s) which form the line of weakness 120. Additionally, it is worth noting that as the nonwoven article 10 preferably comprise an aqueous antimicrobial composition, e.g., hydrogen peroxide, the recloseable access flap 115 should be configured to reduce the evaporation of the aqueous composition from the loaded substrates 10 within the package 75.

The two ends of the line of weakness 120 of the present invention may be connected by a hinge line to forms recloseable access flap 115. The hinge line may allow the recloseable access flap 115 to be biased away from the package 75 such that the interior contents of the package 75 can be accessed.

As shown in FIG. 7, the nonwoven articles 10 may be in an orientation within the package 75 in which the nonwoven article 10 driver end 130 may be adjacent to the left side panel 100 and the nonwoven article 10 operator side 125 may be adjacent to the right-side panel 102 or vice versa. As illustrated in FIG. 7, a portion of the package 75 has been removed to illustrate the location of the driver end 130 of each nonwoven article 10.

While any suitable package may be utilized, as the nonwoven articles 10 may comprise an aqueous composition, inhibiting evaporation of the aqueous composition from the nonwoven articles 10 can ensure a better use experience by the user. An exemplary material for the package of the substrates 10 of the present disclosure is a plastic film which has sufficient thickness to inhibit evaporation of the aqueous antimicrobial composition from the nonwoven articles 10. Such packages should comprise a plastic material which is compatible with the antimicrobial composition, e.g., aqueous hydrogen peroxide. In such forms, the plastic material may be selected from at least one of the following: polyethylene, polyethylene terephthalate, polypropylene, polyvinylalcohol or a mixture thereof.

However, where alternatives to plastic are desired, a paper-based package may be utilized. By paper-based material, we herein mean a material comprising paper. Without wishing to be bound by theory, by ‘paper’ we herein mean a material made from a cellulose-based pulp. Preferably, the paper-based material comprises paper, cardboard, or a mixture thereof, wherein preferably, cardboard comprises paper-board, corrugated fiber-board, or a mixture thereof. The paper-based material may comprise a printed image thereon.

As noted herein, the antimicrobial composition can be aqueous. To ensure that the paperboard package withstands the aqueous composition, an interior surface of the paper-board packaging may comprise a liner having liquid barrier properties. For example, the liner may comprise a plastic material comprising at least one of: polyethylene, polyethylene terephthalate, polypropylene, polyvinylalcohol or a mixture thereof. The barrier material may be a biaxially orientated polypropylene, a metallised polyethylene terephthalate or a mixture thereof. Alternatively, the second material may be a wax, a cellulose material, polyvinylalcohol, or a mixture thereof. It is worth noting that in some configurations, the second material may be discrete from the paper-based material, e.g., an insert into the paper-based material.

Preferably, the paper-based laminate comprises greater than 50%, preferably greater than 85%, and more preferably greater than 95% by weight of the laminate of paper-based materials while the plastic material comprises the remaining weight percentage. Preferably, the plastic material has a thickness of between 10 micron and 40 micron, more preferably between 10 micron and 35 micron. As noted, the loaded substrates may comprise aqueous hydrogen peroxide. Accordingly, the second material should be selected to minimize the evaporation of the aqueous hydrogen peroxide from the loaded substrates as well as minimize the leakage of the aqueous hydrogen peroxide to the paper-based material to avoid weakening of the overall package.

Additional paper-based packages are described in U.S. Pat. Nos. 11,821,142; 11,913,173; 11,913,174; and US2022/0033158. The paper-based packages described herein may be recyclable in paper recycling streams which reduces the amount of material sent to landfill.

### Process

A process for sanitizing or providing an antimicrobial benefit to fabrics can be as set forth herein. The process can comprise the steps of grasping a discrete nonwoven article 10 having a frangible boundary, tearing the nonwoven article 10 along the frangible boundary 30 into a first portion 20 and a second portion 25, placing one or both of the first portion 20 and second portion 25 inside an automatic laundry drying machine containing fabric articles, and operating the automatic laundry machine. The nonwoven article 10 may be pulled from the package 75 where the nonwoven article 10 is in a stack 70. The nonwoven article 10 may be placed inside the automatic laundry drying machine without tearing the nonwoven article 10 along the frangible boundary 30 if the laundry load fills more than half of the drum of the automatic laundry drying machine. Either the first portion 20 or the second portion 25 may be placed without the other portion inside the automatic laundry drying machine if the laundry load fills less than half of the drum. Placing both the first portion 20 and the second portion 25 inside the automatic laundry drying machine when the laundry load fills more than half of the drum can provide the benefit of more of the individual fabric articles in this larger sized load acquiring more of the antimicrobial composition, perfume, and/or optional ingredients because there is a greater quantity of antimicrobial composition, perfume, and/or optional ingredients available to be dispersed. Having the full nonwoven article 10, or a portion of the nonwoven article 10, depending on the quantity of clothing in the laundry load, may allow for more accurate dosing of the antimicrobial composition, perfume, and/or optional ingredients, and may lead to less situations of underdosing. A more accurate dosing of the antimicrobial composition, perfume, and/or optional ingredients may result in the benefits of softer, fresher smelling clothing, with less static cling. It is worth noting that for fabric article loads which are greater than half the drum, more than one loaded substrate may be utilized.

Manufacturers of the nonwoven articles of the present disclosure may provide the loaded nonwoven articles for sale or may provide a kit in which a consumer may load the nonwoven articles themselves, e.g. dry nonwoven articles sold in conjunction with or recommended to be used with an aqueous antimicrobial composition. Manufacturers can instruct the consumer to follow one or more of the steps discussed herein regarding the use of the nonwoven articles of the present disclosure.

As noted previously, the nonwoven articles of the present disclosure may be applied to one or more fabric articles as a spot treatment either prior to the one or more fabric articles being placed in the dryer or after drying, preferably prior to being placed into the dryer. In such methods, particularly where the nonwoven article comprises a frangible boundary between the first portion and the second portion, the first portion may be separated from the second portion and be utilized for spot treatment while the second portion is placed into the dryer with the one or more fabric articles.

Additionally, it is contemplated where the nonwoven articles of the present disclosure may be provided as a kit. In such configurations, the nonwoven articles may be sold in the dry state without the aqueous antimicrobial composition. The aqueous antimicrobial composition may be sold in a separate container. The separate container may be packaged together with the nonwoven articles or packaged separately therefrom.

Such configurations can encourage the use and re-use of a liquid/vapor resistant container. For example, the consumer could place the dry nonwoven articles into the reusable container and then add the composition into the reusable container as well. These steps could easily be swapped as well.

### Additional Examples

Example A: A method of producing antimicrobial vapor in a laundry dryer comprising the steps of: placing a nonwoven article in the laundry dryer, wherein the nonwoven article comprises from about 8 grams to about 10 grams of aqueous antimicrobial composition thereon; placing one or more fabric articles which are desired to be disinfected in the laundry dryer; and operating the laundry dryer in a first cycle, wherein the first cycle has a time period of at least about 15 minutes and an operating temperature of at least about 60 °C.

Example B: The method of Example A, wherein the first cycle time period is from between about 15 minutes to about 40 minutes, more preferably from about 15 minutes to about 30 minutes, or even more preferably from about 15 minutes to about 25 minutes.

Example C: The method of Example A, wherein the first cycle time period is about 20 minutes.

Example D: The method of any of Examples A-C, wherein the antimicrobial composition is at a concentration of from about 2.5 percent to about 7.5 percent, more preferably from about 3 percent to about 6 percent, or even more preferably from about 4 percent to about 5 percent.

Example E: The method of any of the Examples A-D, wherein the antimicrobial composition comprises hydrogen peroxide.

Example F: The method of any of Examples A-E, wherein log reduction of antimicrobial reactive materials is at least 2, more preferably at least 3, even more preferably at least 4, or most preferably at least 5.

Example G: The method of Example F, wherein the log reduction of antimicrobial reactive materials is from between about 5 to about 6.

Example H: The method of any of Examples A-G, wherein the first cycle operating temperature is from about 60 °C to about 80 °C, more preferably from about 65 °C to about 75 °C.

Example I: The method of Example H, wherein nonwoven article comprises a plurality of fibers and/or filaments which have reduced and preferably no reaction with the plurality of fibers and/or filaments that maintain peroxide loading and concentrations for at least 3 months, more preferably 6 months, or most preferably for over 1 year.

Example J: The method of any of Examples A-I, wherein the substrate is loaded with 5 percent aqueous hydrogen peroxide.

Example K: The method of Example J, wherein hydrogen peroxide vapor is delivered at a concentration of from about 10 ppm to about 35 ppm, preferably from about 12 ppm to about 25 ppm, or even more preferably from about 15 ppm to about 20 ppm.

Example L: The method of any of Examples A-K, further comprising the step of: spot treating one or more fabric articles by applying the nonwoven article or a portion thereof to the one or more fabric articles.

Example M: The method of any of Examples A-K, wherein the loaded substrate comprises a first portion and a second portion with a frangible boundary therebetween and wherein the method further comprises the steps of spot treating one or more fabric articles by applying the first portion of the loaded substrate to the one or more articles and separating the first portion from the second portion.

Example N: The method of Example M, further comprising the step of placing the second portion into the dryer.

### Further Definitions and Cross-References

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of producing antimicrobial vapor in a laundry dryer comprising the steps of:
placing a nonwoven article in the laundry dryer, wherein the nonwoven article comprises from about 8 grams to about 10 grams of aqueous antimicrobial composition thereon;
placing one or more fabric articles which are desired to be disinfected in the laundry dryer; and
operating the laundry dryer in a first cycle, wherein the first cycle has a time period of at least about 15 minutes and an operating temperature of at least about 60 °C.

2. The method of claim 1, wherein the first cycle time period is from between about 15 minutes to about 40 minutes.

3. The method of claim 1, wherein the first cycle time period is from between from about 15 minutes to about 25 minutes.

4. The method of claim 1, wherein the first cycle time period is about 20 minutes.

5. The method of claim 1, wherein the antimicrobial composition is at a concentration of from about 2.5 percent to about 7.5 percent.

6. The method of claim 1, wherein the antimicrobial composition is at a concentration of from about 3 percent to about 6 percent.

7. The method of claim 1, wherein the antimicrobial composition is at a concentration of from about 4 percent to about 5 percent.

8. The method of claim 2, wherein the antimicrobial composition comprises hydrogen peroxide.

9. The method of claim 8, wherein log reduction of antimicrobial reactive materials is at least 2.

10. The method of claim 8, wherein log reduction of antimicrobial reactive materials is at least 4.

11. The method of claim 8, wherein the log reduction of antimicrobial reactive materials is from between about 5 to about 6.

12. The method of claim 2, wherein the first cycle operating temperature is from about 60 °C to about 80 °C.

13. The method of claim 2, wherein the first cycle operating temperature is from about 65 °C to about 75 °C.

14. The method of claim 8, wherein the nonwoven article comprises a plurality of fibers and/or filaments which have reduced or no reaction with the hydrogen peroxide concentrations for at least 3 months.

15. The method of claim 8, wherein the nonwoven article comprises a plurality of fibers and/or filaments which have reduced or no reaction with the hydrogen peroxide concentrations for at least 1 year.

16. The method of claim 1, wherein the substrate is loaded with 5 percent aqueous hydrogen peroxide.

17. The method of claim 16, wherein hydrogen peroxide vapor is delivered at a concentration of from about 10 ppm to about 35 ppm.

18. The method of claim 1, further comprising the step of: spot treating one or more fabric articles by applying the nonwoven article or a portion thereof to the one or more fabric articles.

19. The method of claim 1, wherein the loaded substrate comprises a first portion and a second portion with a frangible boundary therebetween and wherein the method further comprises the steps of spot treating one or more fabric articles by applying the first portion of the loaded substrate to the one or more articles and separating the first portion from the second portion.

20. The method of claim 19, further comprising the step of placing the second portion into the dryer.
